(19) United States
(12) Patent Application Publication
(10) Pub. No.: US 2019/0029888 A1
(43) Pub. Date: Jan. 31, 2019

(54) Rubber Bandage for Finger

(71) Applicant: Mary Coffman, St Joseph, MO (US)

(72) Inventor: Mary Coffman, St Joseph, MO (US)

(21) Appl. No.: 15/661,178

(22) Filed: Jul. 27, 2017

(51) Int. Cl. A61F 13/10; A61F 13/00

(52) U.S. Cl. CPC A61F 13/105; A61F 13/00017; A61F 2013/00293; A61F 13/00063; A61F 2013/00093; A61F 13/00038

(57) ABSTRACT

The current invention is bandage that provides increased security of medical gauze and antibiotic ointment for wounds on fingers. The gauze is on the interior of the bandage, and is used to cover a finger wound. Antibiotic ointment is on the gauze to promote a healthy healing process. The exterior is a rubber tube, which comes in a variety of sizes to allow a secure fit. The tube is composed of a hypoallergenic rubber.

### CROSS-REFERENCES TO RELATED APPLICATIONS (IF ANY)

None.

### BACKGROUND

### 1. Field of the Invention

This invention relates to bandages, particularly ones designed to keep the bandage in place on a finger.

### 2. Description of Prior Art

The market for wound care products is becoming more competitive as manufacturers are finding ways to take hospital-grade wound care treatment and make them accessible as over-the-counter options. Further the largest manufacturers have intensive research and development capital to find new more cost-effective ways to make products that are biodegradable or help regenerate skin in wounded areas faster.

The current art for wound care comprises of gauze (thin strips of fabric, such as linen or cotton), medical tape (adhesive tape used to hold bandage in place), plasters (this appears to be the generic term for “Band-aid”), first aid kits, and “New Skin” (‘liquid bandage, apply the liquid and the wound will be sealed together’).

There are a growing number of consumers that have allergies to the adhesive glue on traditional adhesive bandages and are seeking other alternatives.

There is still room for improvement in the art.

### SUMMARY OF THE INVENTION

The current invention is bandage designed to stay in place to cover a finger wound and keep moisture out. The design consists of a rubber tube containing a gauze bandage. The rubber tube is created in multiple sizes, to fit snugly around a finger. The product works by putting the wounded finger through the tube and adjusting the tube until the gauze covers the wound. When properly sized and placed, the bandage will stay in place and keep out moisture better than current flexible band bandages.

The bandages will come in various colors, sizes, and designs.

### BRIEF DESCRIPTION OF THE DRAWINGS

Without restricting the full scope of this invention, the preferred form of this invention is illustrated in the following drawings:

FIG. 1 shows a bandage on a finger covering the middle of the top side of the finger;

FIG. 2 shows a front view of the bandage;

FIG. 3 displays a side view of the bandage;

FIG. 4 shows top view of the bandage; and

FIG. 5 displays a bottom view of the bandage.

### DESCRIPTION OF THE PREFERRED EMBODIMENT

There are a number of significant design features and improvements incorporated within the invention.

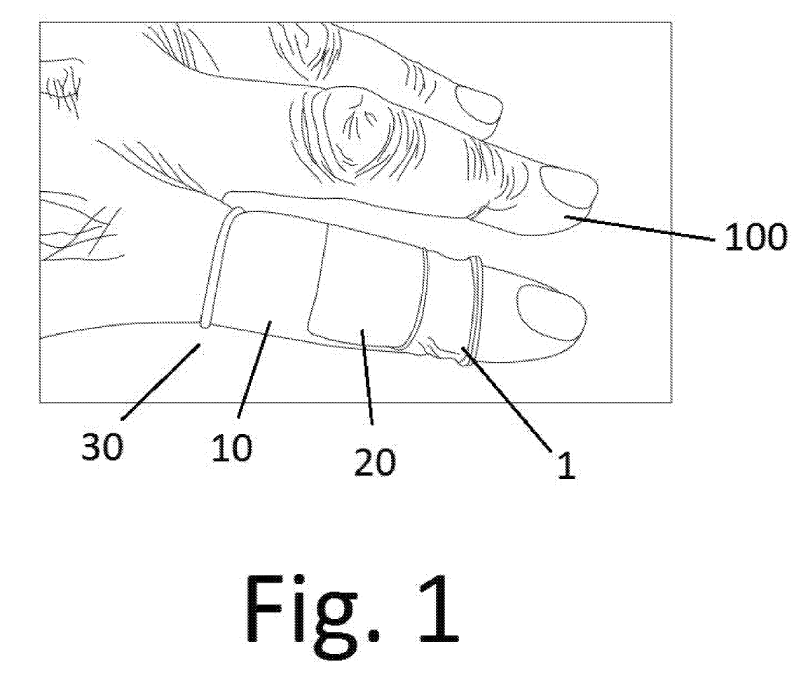

The current invention is bandage 1 designed to stay in place to cover a finger wound and keep moisture out. The bandage 1 is an innovative wound care product that is specifically developed for wounds on fingers 100 as shown in FIG. 1. The bandage 1 stays attached at all times and prevents moisture from getting to the wound.

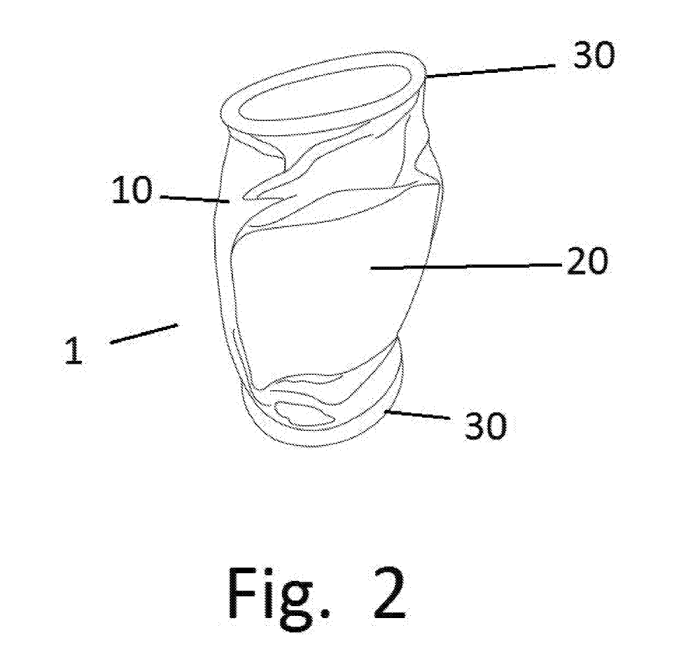

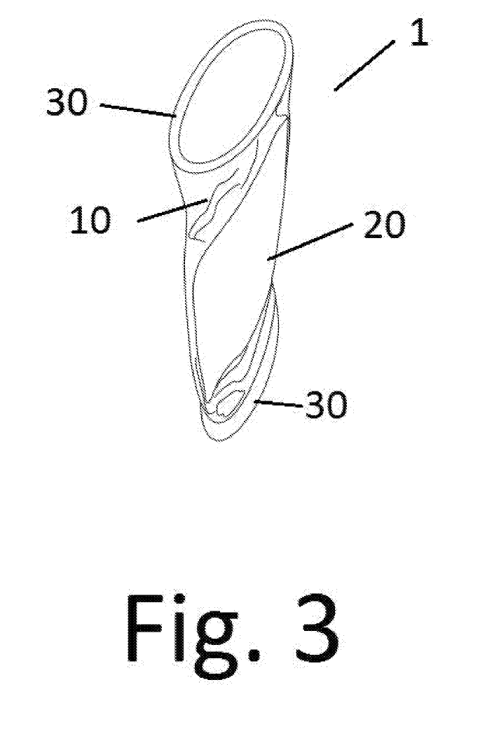

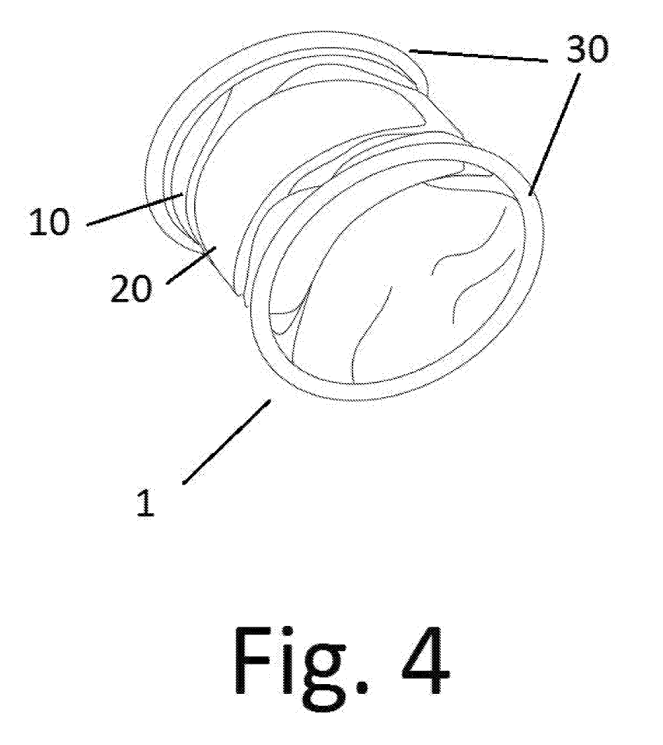

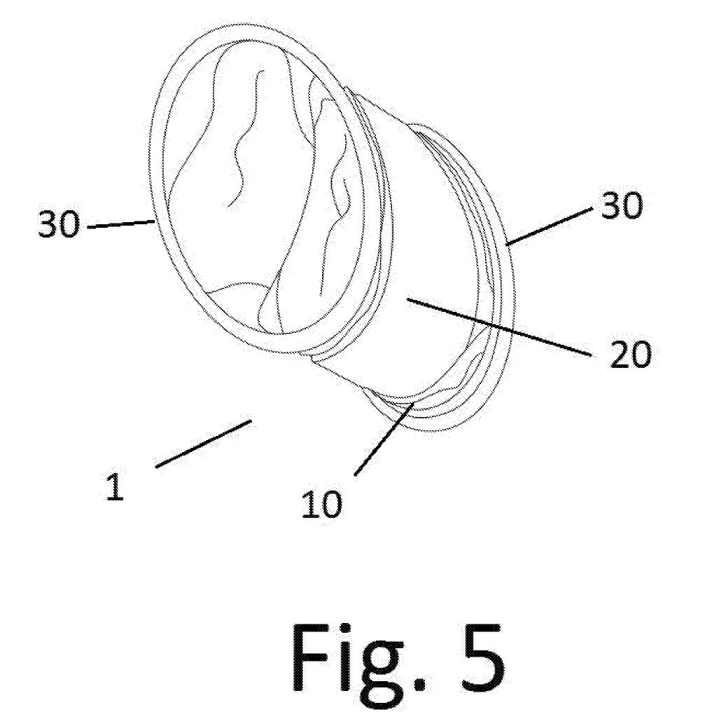

As shown in FIGS. 2 through 5, the bandage design consists of a rubber tube 10 containing a gauze bandage 20 secured inside of the tube 10. The rubber tube 10 can be created in multiple sizes depending on the finger 100 size so that it fits snugly around a finger 100. The bandage 1 has two elastic bands 30 at each end of the tube 10 to assist in securing the bandage 1 to the finger 100.

### Operation

The product works by putting the wounded finger 100 through the tube 10 and adjusting the tube 10 until the gauze 20 covers the wound. When properly sized and placed, the bandage 1 will stay in place and keep out moisture better than current flexible band bandages.

The Internal gauze 20 can be lined with an antibiotic ointment to speed up the healing process.

The bandage 1 is made of a flexible, hypoallergenic rubber in the preferred embodiment.

### Advantages

The innovative bandage can be used by more people, is more secure, and provides an improved healing process. The bandage is use-able by a larger segment of the population, as it is made of hypoallergenic rubber. Being a rubber tube, it lacks any adhesives for application; this allows the product to be more secure, especially in wet conditions allowing the user to get the finger wet including swimming. The gauze has an antibiotic ointment to improve healing.

Although the present invention has been described in considerable detail with reference to certain preferred versions thereof, other versions are possible such as different tension means, bobber shapes and sizes and different colored lights. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided. With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

That which is claimed is:

1. A bandage comprising:
An elastic tube with a gauze bandage inside of the tube.

2. A bandage according to claim 1 comprising:
Having elastic bands at the end of the tube.

3. A bandage according to claim 1 comprising:
Having the gauze lined with an antibiotic ointment.

4. A bandage according to claim 1 comprising:
Having the tube made of an hypoallergenic rubber.

5. A bandage according to claim 1 comprising:
Having the tube made in multiple sizes,

6. A process to bandage a finger using the bandage of claim 1.
